SEQUENCE LISTING (63 sequences)

SEQ ID NO: 1 - DNA, 536 bp, Artificial
  Synthetic DNA sequence from promoter of inhA gene from B. megaterium
gagctggaaaagaaagggaaatccgtgagttatgaaggggatttcttgtttaaaattgatgaaaaaccttatgaaatcgcagcgcgcaatatgtctatttggagcatgtcgctctatcatatcttgctgcagcgtcaaaaagaacattcttaaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttact

SEQ ID NO: 2 - DNA, 541 bp, Artificial
  Synthetic DNA sequence from inhA promoter
gagctggaaaagaaagggaaatccgtgagttatgaaggggatttcttgtttaaaattgatgaaaaaccttatgaaatcgcagcgcgcaatatgtctatttggagcatgtcgctctatcatatcttgctgcagcgtcaaaaagaacattcttaaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttactgtaca

SEQ ID NO: 3 - DNA, 522 bp, Artificial
  Synthetic DNA sequence from inhA promoter
gagctggaaaagaaagggaaatccgtgagttatgaaggggatttcttgtttaaaattgatgaaaaaccttatgaaatcgcagcgcgcaatatgtctatttggagcatgtcgctctatcatatcttgctgcagcgtcaaaaagaacattcttaaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaa

SEQ ID NO: 4 - DNA, 349 bp, Artificial
  Synthetic DNA sequence of truncated region from promoter of inhA gene from B. megaterium
ttatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaa

SEQ ID NO: 5 - DNA, 249 bp, Artificial
  Synthetic DNA sequence of truncated region from promoter of inhA gene from B. megaterium
ctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaa

SEQ ID NO: 6 - DNA, 199 bp, Artificial
  Synthetic DNA sequence of truncated region from promoter of inhA gene from B. megaterium
tttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaa

SEQ ID NO: 7 - DNA, 124 bp, Artificial
  Synthetic DNA sequence of truncated region from promoter of inhA gene from B. megaterium
actataaaactattttacattatctactagaatcaaaaaattttaataatatagtaaaatgtagttgaccttcctagttttagaatataaaataaatcttgctaatagtttgaaaattctaaaa

SEQ ID NO: 8 - DNA, 2624 bp, Artificial
  Synthetic DNA sequence of inhA/celA construct
gagctggaaaagaaagggaaatccgtgagttatgaaggggatttcttgtttaaaattgatgaaaaaccttatgaaatcgcagcgcgcaatatgtctatttggagcatgtcgctctatcatatcttgctgcagcgtcaaaaagaacattcttaaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttactgtacaatgaagacgaagtggctaatatcagtcataatcctatttgttttcatttttcctcaaaatctagtttttgctggtactagtagtacggcaatcgcacaggaaggagcagctccggccgctatattacatccagagaaatggcctcgacctgcgacacaacgacttattgacccggcagttgaaaaaagagttgatgctctgttaaaacagttatctgttgaagaaaaagtagggcaagttatacagggtgatattgggacaattacaccagaagacctgcgcaaatatccactaggttctattttagccggaggaaacagcggcccgaatggagatgatcgtgctcctccaaaggagtggcttgatctagctgatgctttttaccgtgtaagtttagaaaaacggccaggccataccccgataccagtgctttttggcattgatgcagttcatggacatggcaatatcgggtctgcgacaattttccctcacaatattgcacttggagcaacccatgatccagaacttctacgaagaattggtgaggtaacagctgttgaaatggctgctacgggaattgattggacatttgcgcctgcactgtctgttgtgagagatgatcgatggggacgaacatatgaaggcttctcagaagatccagaaattgtagctgcgtattcagcagcaattgtggaaggcgtacagggtaaatttggttctaaggattttatggcgccgggtcgcattgtagcgtcagcaaagcacttcttagctgatggtggaacagatcaaggacgcgatcagggagatgcacgcatttcagaagacgaactaattcgcattcataatgctggataccctcctgcgattgacgcaggagtgctgacagtaatggcttctttttcatcctggcaggggattaaacaccatggccataaacaacttttaacagatgtattaaaaggacaaatggggtttaatggatttattgtgggggattggaatgctcatgaccaagtaccgggctgtactaaatttaattgtccaacatctcttattgcgggtttagatatgtatatggccgccgattcctggaagcagctgtacgaaaacaccttagcacaagtgaaagatggtactattcctatggcacgtctagatgatgccgtaagacgaatcttgcgagtcaaggtgttggctggcttattcgagaaacctgcgccaaaagatcgtccggggttaccaggccttgaaacactaggatcacctgaacatagagccgtaggccgtgaagctgttcgaaaaagcctagttcttcttaaaaatgataaaggtacccttccactgtcaccaaaggctagagtattagttgcaggtgacggagcagataatattggcaaacagtcggggggctggacgattagttggcaaggaactggaaaccgtaacgatgaatttccgggtgctacatccattttaggtgggattcgagacgctgtagctgatgcaggagggtccgtagaatttgatgtagcgggtcagtataaaacaaaacctgatgtagctattgttgtttttggcgaagaaccttatgctgagtttcagggagatgtggagacactggaatatcaaccagatcaaaaacaagatcttgctctactcaagaaactgaaagatcagggaatacctgttgttgctgttttcctttctggacgaccgatgtgggttaatcctgaacttaatgccagcgatgctttcgttgcagcatggcttcctggcacagaaggtggcggtgtggcggatgtattgtttacagacaaagcgggaaaagtacaacatgattttgcaggaaaattgtcatatagttggccgcgtacggcagcccagacaacagttaaccgtggtgatgcagattataatccgttatttgcgtatggttacggtttaacgtacaaagataaatcgaaagtgggcactctacctgaagaaagtggagtaccggctgaagcgcgacagaatgcagggatttattttcgcgcaggggcgctgagattaccaggaaggtttctgtgatcatgagcat

SEQ ID NO: 9 - protein, 690 aa, Artificial
  Synthetic peptide sequence of translation of inhA/celA construct
MKTKWLISVIILFVFIFPQNLVFAGTSSTAIAQEGAAPAAILHPEKWPRPATQRLIDPAVEKRVDALLKQLSVEEKVGQVIQGDIGTITPEDLRKYPLGSILAGGNSGPNGDDRAPPKEWLDLADAFYRVSLEKRPGHTPIPVLFGIDAVHGHGNIGSATIFPHNIALGATHDPELLRRIGEVTAVEMAATGIDWTFAPALSVVRDDRWGRTYEGFSEDPEIVAAYSAAIVEGVQGKFGSKDFMAPGRIVASAKHFLADGGTDQGRDQGDARISEDELIRIHNAGYPPAIDAGVLTVMASFSSWQGIKHHGHKQLLTDVLKGQMGFNGFIVGDWNAHDQVPGCTKFNCPTSLIAGLDMYMAADSWKQLYENTLAQVKDGTIPMARLDDAVRRILRVKVLAGLFEKPAPKDRPGLPGLETLGSPEHRAVGREAVRKSLVLLKNDKGTLPLSPKARVLVAGDGADNIGKQSGGWTISWQGTGNRNDEFPGATSILGGIRDAVADAGGSVEFDVAGQYKTKPDVAIVVFGEEPYAEFQGDVETLEYQPDQKQDLALLKKLKDQGIPVVAVFLSGRPMWVNPELNASDAFVAAWLPGTEGGGVADVLFTDKAGKVQHDFAGKLSYSWPRTAAQTTVNRGDADYNPLFAYGYGLTYKDKSKVGTLPEESGVPAEARQNAGIYFRAGALRLPGRFL

SEQ ID NO: 10 - DNA, 1912 bp, Artificial
  Synthetic DNA sequence including inhA/cbhII construct
gagctggaaaagaaagggaaatccgtgagttatgaaggggatttcttgtttaaaattgatgaaaaaccttatgaaatcgcagcgcgcaatatgtctatttggagcatgtcgctctatcatatcttgctgcagcgtcaaaaagaacattcttaaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttactgtacaatgaaaaagaaaaaacaggctttaaaggtattattatcagttggtatcctttcttcatcatttgcttttgcacatacgagcagtgccgcgactagtatggggcctgctgcacctactgcacgtgtggataatccttatgtaggcgcgacaatgtacgtaaatccagaatggtcagctcttgctgcttcggaaccaggtggtgatcgtgttgcagatcaacctacggctgtttggttagatcgtattgcaactattgaaggtgttgatggaaaaatgggattacgagaacatcttgatgaagcgttacaacaaaaaggaagcggagaacttgtggtacagttagtaatttatgatttacctggtcgtgattgcgcggctcttgctagtaatggtgaattaggtcctgatgaattagatcgatataaaagcgaatatattgatccgattgcagacattttatcggattccaaatatgaaggacttcgtattgttacggttattgaaccagacagcttacctaatttagtaacaaacgcaggtggtacagatacaacgacagaagcatgtactactatgaaagcaaacggtaattatgaaaaaggggtatcgtatgcgctttctaaattaggtgcaattccgaacgtatacaactatattgatgctgctcatcatggatggttaggatgggacacaaatttagggccatccgtacaggaattttataaagtggcaacatcaaatggcgcatccgttgatgatgtggcgggatttgcagtcaatacagctaattattcacctactgtagaaccttattttacggtttcagatacggtgaatgggcagacggtacgtcaatctaaatgggttgactggaatcaatacgtagatgaacaaagttatgcgcaggctttacgaaacgaagctgtcgccgctggatttaatagcgatattggtgtgattattgatacatcccgaaatggatggggtggttcagatcgcccttcagggcctggccctcaaacttccgtagatgcttatgtagatggatcacgaattgatcgtcgcgttcatgtaggaaattggtgtaatcagtctggagcaggcttaggtgaaagaccaacagcagcaccagctagcgggattgatgcatatacatggattaaaccgccgggcgaatctgatggaaattcagctccggttgataatgacgaaggaaaaggatttgaccaaatgtgtgatcctagctaccagggaaacgctcgcaatggctacaatccttcaggagcgttacctgatgcaccattaagtggacaatggttttcggcacaatttcgtgaattaatgcaaaatgcatatcctccattatcttga

SEQ ID NO: 11 - protein, 456 aa, Artificial
  Synthetic peptide sequence of translation of inhA/cbhII construct
MKKKKQALKVLLSVGILSSSFAFAHTSSAATSMGPAAPTARVDNPYVGATMYVNPEWSALAASEPGGDRVADQPTAVWLDRIATIEGVDGKMGLREHLDEALQQKGSGELVVQLVIYDLPGRDCAALASNGELGPDELDRYKSEYIDPIADILSDSKYEGLRIVTVIEPDSLPNLVTNAGGTDTTTEACTTMKANGNYEKGVSYALSKLGAIPNVYNYIDAAHHGWLGWDTNLGPSVQEFYKVATSNGASVDDVAGFAVNTANYSPTVEPYFTVSDTVNGQTVRQSKWVDWNQYVDEQSYAQALRNEAVAAGFNSDIGVIIDTSRNGWGGSDRPSGPGPQTSVDAYVDGSRIDRRVHVGNWCNQSGAGLGERPTAAPASGIDAYTWIKPPGESDGNSAPVDNDEGKGFDQMCDPSYQGNARNGYNPSGALPDAPLSGQWFSAQFRELMQNAYPPLS

SEQ ID NO: 12 - DNA, 38 bp, Artificial
  Synthetic DNA sequence of primer for inhA promoter
ccttcatatgagctggaaaagaaagggaaatccgtgag

SEQ ID NO: 13 - DNA, 40 bp, Artificial
  Synthetic DNA sequence of primer for inhA promoter
ggtatgtacagtaaacccctccattttagaattttcaaac

SEQ ID NO: 14 - DNA, 39 bp, Artificial
  Synthetic DNA primer for signal peptide NprM
tactgtacaatgaaaaagaaaaaacaggctttaaaggta

SEQ ID NO: 15 - DNA, 40 bp, Artificial
  Synthetic DNA primer for signal peptide NprM
ccatactagtcgcggcactgctcgtatgtgcaaaagcaaa

SEQ ID NO: 16 - DNA, 40 bp, Artificial
  Synthetic DNA primer for signal peptide PenG
ttactgtacaatgaagacgaagtggctaatatcagtcata

SEQ ID NO: 17 - DNA, 40 bp, Artificial
  Synthetic DNA primer for signal peptide PenG
tactactagtaccagcaaaaactagattttgaggaaaaat

SEQ ID NO: 18 - DNA, 36 bp, Artificial
  Synthetic DNA primer for construction of truncated promoter in plasmid BmI201
aattaacatatgtttgcatgtgtaagaagggtttat

SEQ ID NO: 19 - DNA, 36 bp, Artificial
  Synthetic DNA primer for construction of truncated promoter in plasmid BmI251
aattaacatatgaaaaatacacttcatatattttta

SEQ ID NO: 20 - DNA, 36 bp, Artificial
  Synthetic DNA primer for construction of truncated promoter in plasmid BmI301
aattaacatatgttttgtttttatataatagtaata

SEQ ID NO: 21 - DNA, 36 bp, Artificial
  Synthetic DNA primer for construction of truncated promoter in plasmid BmI386
aattaacatatgtaaaatttatttgtatacagagat

SEQ ID NO: 22 - DNA, 29 bp, Artificial
  Synthetic DNA primer for construction of truncated promoter
catatgacgatagattttaatggacccaa

SEQ ID NO: 23 - DNA, 201 bp, Artificial
  Synthetic DNA sequence of truncated inhA promoter
tttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttact

SEQ ID NO: 24 - DNA, 251 bp, Artificial
  Synthetic DNA sequence of truncated inhA promoter
aaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttact

SEQ ID NO: 25 - DNA, 301 bp, Artificial
  Synthetic DNA sequence of truncated inhA promoter
ttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttact

SEQ ID NO: 26 - DNA, 386 bp, Artificial
  Synthetic DNA sequence of truncated inhA promoter
taaaatttatttgtatacagagattatcgcttctcgtctattgagaagtgatttttttatctttctgtatccataatggggaattttttgtttttatataatagtaatacggaaaagatagttctaattccctaaaaaaatacacttcatatatttttagttttggttatcgatttggcatggaatttgcatgtgtaagaagggtttatcagacattttaattgtcaggaaaagaaaactagttacatactataaaactattttacattatctactagaatcaaaaaattttaataatatagtaaaatgtagttgaccttcctagttttagaatataaaataaatcttgctaatagtttgaaaattctaaaatggaggggtttact

SEQ ID NO: 27 - DNA, 60 bp, Artificial
  Synthetic DNA sequence of variant of inhA promoter
tataaaataaggcatgctaatagtttgaaaatttaaaatggaggggcgaagtgtacaatg

SEQ ID NO: 28 - DNA, 61 bp, Artificial
  Synthetic DNA sequence of variant of inhA promoter
tataaaataaggcatgctaatagtttgaaaattctaaaatggaggggctcgatgtacaatg

SEQ ID NO: 29 - DNA, 61 bp, Artificial; identical to SEQ ID NO: 28
  Synthetic DNA sequence of variant of inhA promoter

SEQ ID NO: 30 - DNA, 61 bp, Artificial
  Synthetic DNA sequence of variant of inhA promoter
tataaaataaggcatgctaatagtttgaaaattctaaaatggaggggtttatggaccaatg

SEQ ID NO: 31 - DNA, 61 bp, Artificial
  Synthetic DNA sequence of variant of inhA promoter
tataaaataaggcatgctaatagtttgaaaattctaaaatggaggggcattatgtacaatg

SEQ ID NO: 32 - DNA, 61 bp, Artificial
  Synthetic DNA sequence of variant of inhA promoter
tataaaataaggcatgctaatagtttgaaaattctaaaatggaggggtcctatgtacaatg

SEQ ID NO: 33 - DNA, 56 bp, Artificial
  Synthetic DNA sequence of variant of inhA promoter
tataaaataaggcatgctaatagtttgaaaatttaagagatggagggattcgtatg

SEQ ID NO: 34 - DNA, 56 bp, Artificial
  Synthetic DNA sequence of variant of inhA promoter
tataaaataaggcatgctaatagtttgaaaatttgatatttggagggaataaaatg

SEQ ID NO: 35 - DNA, 56 bp, Artificial
  Synthetic DNA sequence of variant of inhA promoter
tataaaataaggcatgctaatagtttgaaaattatgtaggagggccatactctatg

SEQ ID NO: 36 - DNA, 61 bp, Artificial
  Synthetic DNA sequence of variant of inhA promoter
tataaaataaggcatgctaatagtttgaaaattctaaaatggaggggtttatgggacaatg

SEQ ID NO: 37 - DNA, 61 bp, Artificial
  Synthetic DNA sequence of variant of inhA promoter
tataaaataaggcatgctaatagtttgaaaattctaaaatggaggggcaacatgtacaatg

SEQ ID NO: 38 - DNA, 61 bp, Artificial
  Synthetic DNA sequence of variant of inhA promoter
tataaaataaggcatgctaatagtttgaaaattctaaaatggaggggtcttatgtacaatg

SEQ ID NO: 39 - DNA, 61 bp, Artificial
  Synthetic DNA sequence of variant of inhA promoter
tataaaataaggcatgctaatagtttgaaaattctaaaatggaggggtttactccatgatg

SEQ ID NO: 40 - DNA, 61 bp, Artificial
  Synthetic DNA sequence of variant of inhA promoter
tataaaataaggcatgctaatagtttgaaaattctaaaatggaggggtttactgtacaatg

SEQ ID NO: 41 - DNA, 61 bp, Artificial
  Synthetic DNA sequence of variant of inhA promoter
tataaaatataggctgctaatagtttgaaaattctaaaatggaggggtttactgtacaatg

SEQ ID NO: 42 - DNA, 61 bp, Artificial
  Synthetic DNA sequence of variant of inhA promoter
tataaaataaatcttgctaatagtttgaaaattctaaaatggaggggtttactgtacaatg

SEQ ID NO: 43 - DNA, 61 bp, Artificial
  Synthetic DNA sequence of variant of inhA promoter
tataaaatagggtctgctaatagtttgaaaattctaaaatggaggggtttactgtacaatg

SEQ ID NO: 44 - DNA, 56 bp, Artificial
  Synthetic DNA sequence of variant of inhA promoter
tataaaataaatcaatagtttgaaaattctaaaatggaggggtttactgtacaatg

SEQ ID NO: 45 - DNA, 61 bp, Artificial
  Synthetic DNA sequence of variant of inhA promoter
tataaaataatgactgctaatagtttgaaaattctaaaatggaggggtttactgtacaatg

SEQ ID NO: 46 - DNA, 61 bp, Artificial
  Synthetic DNA sequence of variant of inhA promoter
tataaaataaaggcggctaatagtttgaaaattctaaaatggaggggtttactgtacaatg

SEQ ID NO: 47 - DNA, 61 bp, Artificial
  Synthetic DNA sequence of variant of inhA promoter
tataaaataaatctgcctcatagtttgaaaattctaaaatggaggggtttactgtacaatg

SEQ ID NO: 48 - DNA, 61 bp, Artificial
  Synthetic DNA sequence of variant of inhA promoter
tataaaataaatctattttatagtttgaaaattctaaaatggaggggtttactgtacaatg

SEQ ID NO: 49 - DNA, 61 bp, Artificial
  Synthetic DNA sequence of variant of inhA promoter
tataaaataaatctcccctatagtttgaaaattctaaaatggaggggtttactgtacaatg

SEQ ID NO: 50 - DNA, 61 bp, Artificial
  Synthetic DNA sequence of variant of inhA promoter
tataaaatacggggtgctaatagtttgaaaattctaaaatggaggggtttactgtacaatg

SEQ ID NO: 51 - DNA, 61 bp, Artificial
  Synthetic DNA sequence of variant of inhA promoter
tataaaataatcgatgctaatagtttgaaaattctaaaatggaggggtttactgtacaatg

SEQ ID NO: 52 - DNA, 61 bp, Artificial
  Synthetic DNA sequence of variant of inhA promoter
tataaaataaatctcctgtatagtttgaaaattctaaaatggaggggtttactgtacaatg

SEQ ID NO: 53 - DNA, 61 bp, Artificial
  Synthetic DNA sequence of variant of inhA promoter
tataaaataaatcttcgccatagtttgaaaattctaaaatggaggggtttactgtacaatg

SEQ ID NO: 54 - DNA, 60 bp, Artificial
  Synthetic DNA sequence of variant of inhA promoter
tataaaataaatctccccaatagtttgaaaattctaaatggaggggtttactgtacaatg

SEQ ID NO: 55 - DNA, 61 bp, Artificial
  Synthetic DNA sequence of variant of inhA promoter
tataaaatactcagtgctaatagtttgaaaattctaaaatggaggggtttactgtacaatg

SEQ ID NO: 56 - DNA, 61 bp, Artificial; identical to SEQ ID NO: 55
  Synthetic DNA sequence of variant of inhA promoter

SEQ ID NO: 57 - DNA, 60 bp, Artificial
  Synthetic DNA sequence of variant of inhA promoter
tataaaataaatcttccaatagtttgaaaattctaaaatggaggggtttactgtacaatg

SEQ ID NO: 58 - DNA, 61 bp, Artificial
  Synthetic DNA sequence of variant of inhA promoter
tataaaatattcggtgctaatagtttgaaaattctaaaatggaggggtttactgtacaatg

SEQ ID NO: 59 - DNA, 60 bp, Artificial
  Synthetic DNA sequence of variant of inhA promoter
tataaaatacaactgctaatagtttgaaaattctaaaatggaggggtttactgtacaatg

SEQ ID NO: 60 - DNA, 61 bp, Artificial
  Synthetic DNA sequence of variant of inhA promoter
tataaaataatccgtgctaatagtttgaaaattctaaaatggaggggtttactgtacaatg

SEQ ID NO: 61 - DNA, 61 bp, Artificial
  Synthetic DNA sequence of variant of inhA promoter
tataaaataaatctccttgatagtttgaaaattctaaaatggaggggtttactgtacaatg

SEQ ID NO: 62 - DNA, 61 bp, Artificial
  Synthetic DNA sequence of variant of inhA promoter
tataaaatacacattgctaatagtttgaaaattctaaaatggaggggtttactgtacaatg

SEQ ID NO: 63 - DNA, 61 bp, Artificial
  Synthetic DNA sequence of variant of inhA promoter
tataaaataccaattgctaatagtttgaaaattctaaaatggaggggtttactgtacaatg

(19) United States
(12) Patent Application Publication
(10) Pub. No.: US 2010/0267088 A1
(43) Pub. Date: Oct. 21, 2010

(54) INHA PROMOTERS

(75) Inventors: Jeffrey S. Pollack, Redwood City, CA (US); Oscar Alvizo, San Mateo, CA (US); David J. Galgoczy, San Francisco, CA (US); Dayal Saran, Sunnyvale, CA (US)

Correspondence Address: Codexis, Inc., 200 Penobscot Drive, Redwood City, CA, 94063, US

(73) Assignee: CODEXIS, INC., REDWOOD CITY, CA (US)

(21) Appl. No.: 12/760,827

(22) Filed: Apr. 15, 2010

Related U.S. Application Data
(60) Provisional application No. 61/169,848, filed on Apr. 16, 2009.

(51) Int. Cl. C12P 21/00; C07H 21/04; C12N 15/63; C12N 1/21; C12N 9/00

(52) U.S. Cl. 435/69.1; 536/24.1; 435/320.1; 435/252.31; 435/252.33; 435/183

(57) ABSTRACT

The present invention relates to an isolated DNA sequence having promoter activity and to vectors comprising the DNA sequence. The promoters of the invention are referred to inhA promoters. The invention also relates to a method of producing a protein in a recombinant host cell wherein the host cell comprises a vector comprising an inhA promoter and a coding sequence encoding a protein of interest.

### CROSS-REFERENCE TO RELATED APPLICATIONS

This application claims the benefit pursuant to 35 U.S.C. §119(e) of U.S. Ser. No. 61/169,848 filed Apr. 16, 2009, which is incorporated herein by reference in its entirety.

### FIELD OF THE INVENTION

The present invention relates to an isolated DNA sequence having promoter activity and to vectors comprising the DNA sequence. The invention also relates to a method of producing a protein in a host cell wherein the host cell comprises a vector comprising a DNA sequence having promoter activity according to the invention and a coding sequence encoding a protein of interest.

### BACKGROUND OF THE INVENTION

In the field of molecular biology researchers frequently look for means of maximizing the expression of heterologous genes in host cells with the goal of producing commercially relevant amounts of a desired protein. Recombinant production of a desired protein is often accomplished by constructing vectors suitable for use in host cells wherein the vector comprises a polynucleotide which codes for a desired protein and wherein the polynucleotide is placed under the control of regulatory elements and in particular regulatory elements referred to as promoters. The vector will be introduced into a host cell under suitable conditions for the expression and production of the desired protein. There are numerous promoters known in the art, however, there is a need for new promoters which control the expression of heterologous genes. The present invention fulfills this and other related needs.

### BRIEF SUMMARY OF THE INVENTION

The present invention has multiple aspects.

In one aspect, the invention relates to an isolated promoter comprising a nucleotide sequence of SEQ ID NO: 1 or SEQ ID NO: 3; b) a truncated nucleotide sequence of SEQ ID NO: 1 or SEQ ID NO: 3; c) a nucleotide sequence having at least 90% sequence identity (e.g., at least 91%, at least 92%, at least 93%, at least 94%, at least 95%, at least 96%, at least 97%, at least 98%, and at least 99% sequence identity) to a) or b); or d) a variant nucleotide sequence or a truncated variant nucleotide sequence of SEQ ID NO: 1, wherein said variant or truncated variant has between 1 and 10 substitutions when aligned with SEQ ID NO: 1 or the truncated sequence thereof. In some embodiments, the isolated promoter will be a truncated subsequence of SEQ ID NO: 1 or SEQ ID NO: 3, wherein the truncated subsequence comprises a nucleotide sequence selected from SEQ ID NO: 4; SEQ ID NO: 5; SEQ ID NO: 6; SEQ ID NO: 7; SEQ ID NO: 23, SEQ ID NO: 24, SEQ ID NO: 25, SEQ ID NO: 26 and a nucleotide sequence having at least 90% sequence identity (e.g., at least 91%, at least 92%, at least 93%, at least 94%, at least 95%, at least 96%, at least 97%, at least 98%, and at least 99% sequence identity) to a sequence thereof. In some embodiments, the promoter will be a variant nucleotide sequence or a truncated variant nucleotide sequence of SEQ ID NO: 1. In some embodiments, the promoter comprises SEQ ID NO: 2 and a truncated sequence or a variant sequence thereof and further sequences having at least 95% sequence identity thereto.

In a further aspect, the invention relates to a vector comprising a DNA sequence comprising a promoter encompassed by the invention operably linked to a heterologous coding sequence. In some embodiments, the coding sequence encodes a protein. In some embodiments, the protein is an enzyme. In some embodiments, the enzyme is a cellulase, a glucoamylase, an amylase, a protease, a phytase, a lipase, an esterase, a xylanase, or a laccase. In some embodiments, the vector further comprises a signal sequence.

In another aspect, the invention relates to a recombinant microbial host cell comprising a vector encompassed by the invention. In some embodiments, the recombinant host cell will be a bacterial host cell. In some embodiments, the bacterial host cell will be a Bacillus or Streptomyces host cell. In some embodiments, the Bacillus will be a B. subtilis, B. licheniformis, B. stearothermophilus, B. pumilus, B. megaterium, B. clausii, or B. amyloliquefaciens. In some embodiments, the Streptomyces will be S. avermitilis, S. coelicolor, S. griseus, or S. lividans.

In yet another aspect, the invention relates to a process for producing a protein in a recombinant microbial host cell which comprises transforming the microbial host cell with a vector encompassed by the invention and culturing the transformed microbial host cell under conditions suitable for the expression of the gene and production of the protein. In some embodiments the protein will be recovered from the culture media.

### BRIEF DESCRIPTION OF THE DRAWINGS

FIGS. 1A-1E:

FIG. 1A illustrates an inhA promoter (SEQ ID NO: 1) which includes the ribosome binding region in bold and underlined and the upstream sequence (SEQ ID NO: 3). The putative-35 region and -10 region are highlighted in bold and italics.

FIG. 1B illustrates SEQ ID NO: 8 which includes an inhA/celA construct incorporated into vector pBmI-PG-celA. The inhA promoter is in italics, the restriction sites are in bold and the signal peptide, PenG is underlined.

FIG. 1C illustrates SEQ ID NO: 9 which includes the translated inhA/celA construct. The signal peptide is underlined.

FIG. 1D illustrates SEQ ID NO: 10 which includes an inhA/cbhII construct incorporated into vector pBmX-NM-cbhII. The inhA promoter is in italics, the restriction sites are in bold and the signal peptide, NprM is underlined.

FIG. 1E illustrates SEQ ID NO: 11 which depicts the translated inhA/cbhII construct. The signal peptide is underlined.

FIGS. 2A-B illustrate: (A) an SDS-PAGE of 7, 23, 26, 28.5 and 30-hour time points from 2 L fermentation of a Bacillus megaterium strain expressing CBHII from the inhA promoter and (B) an SDS-PAGE of 6, 24, 26, 29 and 32.5-hour time points of a 2 L fermentation of B. megaterium expressing CelA from the inhA promoter.

FIG. 3. depicts CelA secreted protein activity as measured by pNPG hydrolysis activity over 5 to 32.5 hours. Hydrolysis (Ab405) is indicated by ⋄ and biomass (g/L) is indicated by □.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in analytical chemistry, cell culture, molecular genetics, organic chemistry and nucleic acid chemistry and hybridization described below are those well known and commonly employed in the art. Unless indicated otherwise, the techniques and procedures as described herein are generally performed according to conventional methods in the art and various general references. See, e.g., Sambrook et al. Molecular Cloning: A Laboratory Manual, 3rd ed. (2001) Cold Spring Harbor Laboratory Press, Cold Spring Harbor, N.Y. and Ausubel, ed., Current Protocols in Molecular Biology, 1990-2008, John Wiley Interscience, which are provided throughout this document.

As used herein, the term “promoter” refers to a polynucleotide sequence, particularly a DNA sequence that initiates and facilitates the transcription of a gene. Usually the promoter is located on the same strand as the gene it controls, in a close proximity upstream of the gene, i.e., towards the 5′ region of the coding strand. Promoters may include DNA sequence elements which insure proper binding and activation of RNA polymerase, influence where transcription will start and affect the level of transcription. The size of the promoter may be variable. While not meant to limit the instant invention, in many cases promoter activity is confined to about 150 base pairs (bp) of sequence in the 5′ direction of the site of transcript initiation. However, sequences out to about 500 by 5′ of a structural gene may be involved in promoter activity. Most promoters have conserved sequence elements at −10 by and −35 by (10 nucleotides and 35 nucleotides respectively 5′ to the site of initiation of transcription). As used herein a promoter of the invention will exhibit promoter activity.

The phrase “exhibiting or having promoter activity” means that a DNA sequence functions as a promoter and that a gene or nucleotide sequence which is operably linked to the promoter DNA sequence has ability and function for producing an expression product (e.g., a desired protein). Promoter activity may be assayed by means known in the art and as further described in the disclosure herein. In addition, the presence of promoter activity can be determined by, for instance, confirmation of expression of the gene product of the gene inside or outside of a transformed host.

The term “inducible promoter” means a promoter which initiates transcription only when the host cell comprising the inducible promoter is exposed to some particular external stimulus. The activity of an inducible promoter may be triggered by chemical, physical, nutritional or cell growth factors. Non-limiting examples of inducing factors include environmental factors (e.g., temperature or light), chemical factors (e.g., IPTG, tetracycline or xylose), nutritional factors (e.g., carbon starvation), or cell growth phase (e.g., exponential growth phase). In some embodiments, the external stimulus may be a lack of a particular stimulus. In some embodiments, the inhA promoters of the invention do not require the addition of certain inducers, such as, but not limited to IPTG, xylose or elevated temperature. In some embodiments, an inducible promoter according to the invention may be a “glucose-starvation inducible promoter”. A glucose-starvation inducible promoter is a promoter that is active when glucose concentrations are low. One advantage of a glucose-starvation inducible promoter is that cells can grow on an alternative carbon source (for example, but not limited to glycerol) and at the same time the promoter remains active.

An inducible promoter is distinguished from a constitutive promoter. A “constitutive promoter” means a promoter by which transcription is performed constantly at a given level irrelevant of the growth conditions. A constitutive promoter is capable of expressing a gene located downstream of the promoter without limitation.

The term “inhA promoter” is used herein to collectively refer to the various promoters encompassed by the invention, including but not limited to a promoter comprising a nucleic acid sequence of SEQ ID NO: 1 or SEQ ID NO: 3, truncations (subsequences) thereto and variants thereof wherein the truncations and variants have at least 90% sequence identity when aligned with SEQ ID NO: 1, SEQ ID NO: 3, SEQ ID NO: 4, SEQ ID NO: 5, SEQ ID NO: 6, SEQ ID NO: 7, SEQ ID NO: 23, SEQ ID NO: 24, SEQ ID NO: 25, or SEQ ID NO: 26.

The term “truncation” or “subsequence” used interchangeably herein means a nucleotide sequence having one or more nucleotides deleted from the 5′ and/or 3′ end of the sequence of SEQ ID NO: 1, wherein the truncation or subsequence has promoter activity (that is the functional part thereof).

The term “variant” with reference to a promoter means an inhA promoter which comprises one or more modifications such as substitutions, additions or deletions of one or more nucleotides. The term “variant” as used herein is recombinant (e.g. that has been engineered to include substitution, additions or deletions of at least 1, 2, 3, 4, 5, 6, 7, 8, 9, or 10 polynucleotides as compared to a reference or parent inhA promoter).

The term “reference promoter” or “parent promoter” means a promoter sequence that is used as a basis for sequence comparison. The reference promoter may be a subset of a larger sequence. Generally a reference sequence is at least 25 nucleotides in length, at least 50 nucleotides in length, at least 100 nucleotides in length, at least 150 nucleotides in length, at least 200 nucleotides in length, at least 250 nucleotides in length, at least 300 nucleotides in length, at least 350 nucleotides in length, at least 400 nucleotides in length, at least 450 nucleotides in length, at least 500 nucleotides in length.

The term “nucleic acid” “nucleotides” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “isolated” as used herein means a compound, protein, cell, nucleic acid sequence or an amino acid sequence that is removed from at least one component with which it is naturally associated.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens or enzymes), wherein the amino acid residues are linked by covalent peptide bonds.

As used herein, the term “introduced” in the context of inserting a nucleic acid sequence into a cell means transfected, transduced or transformed (collectively “transformed”) and includes reference to the incorporation of a nucleic acid sequence into a eukaryotic or prokaryotic cell wherein the nucleic acid is incorporated into the genome of the cell.

As used herein, the term “transformation” refers to a process during which exogenous nucleic acid, such as a recombinant expression cassette, is introduced into a host cell such that the nucleic acid is maintained either as an extra chromosomal element or by integration into the host chromosome, at least for the duration when the exogenous nucleic acid is expressed.

As used herein the term “culturing” refers to growing a population of microbial cells under suitable conditions in a liquid or solid medium. In some embodiments, culturing refers to fermentative bioconversion of a carbon substrate to an end-product.

The term “vector,” as used herein, refers to a recombinant nucleic acid designed to carry a coding sequence of interest to be introduced into a host cell. This term encompasses many different types of vectors, such as cloning vectors, expression vectors, shuttle vectors, plasmids, phage or virus particles, and the like. A typical expression vector may also include, in addition to a coding sequence of interest, elements that direct the transcription and translation of the coding sequence, such as a promoter, enhancer, terminator, and signal sequence.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

As used herein the term “expression” includes any step involved in the production of a polypeptide including but not limited to, transcription, post-transcriptional modification, translation, post-translational modification and secretion.

A “signal sequence” is a DNA sequence that is a component of a polynucleotide that encodes a polypeptide and Which directs the polypeptide through a secretory pathway of a cell in which it is synthesized. The polypeptide is commonly cleaved to remove the secretory peptide during transit through the secretory pathway.

As used herein, the terms “identical” or percent “identity,” in the context of describing two or more polynucleotide sequences, refer to two or more sequences or truncated subsequences that are the same or have a specified percentage of nucleotides that are the same when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of variants of the promoter sequences of this invention, e.g., SEQ ID NO: 3, SEQ ID NO: 1 and its variants, the BLAST and BLAST 2.0 algorithms and the default parameters discussed below are used.

A “comparison window” as used herein, includes reference to a segment of any one of the number of contiguous positions selected from the group consisting of from 20 to 500, usually about 50 to about 300, also about 50 to 250, and also about 100 to about 200 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous,” when used to describe an operably linked pair of promoter and coding sequences, refers to the relationship between the promoter and the coding sequence as one not found in nature. In other words, a heterologous coding sequence and a promoter may be from two different organisms, or a heterologous coding sequence and a promoter may be from the same organism, so long as the particular promoter does not, in any naturally occurring organisms, direct the transcription of the particular coding sequence.

When two elements, e.g., a promoter and a coding sequence, are said to be “operably linked,” it is meant that the juxtaposition of the two allows them to be functionally active relationship. In other words, a promoter is “operably linked” to a coding sequence when the promoter controls the transcription of the coding sequence.

The term “recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

A “host cell” is a cell that has the capability to act as a host and expression vehicle for a vector of the present invention. A transformed host cell includes any progeny thereof.

As used herein “a”, “an”, and “the” include plural references unless the context clearly dictates otherwise.

The term “comprising” and its cognates are used in their inclusive sense; that is, equivalent to the term “including” and its corresponding cognates.

### DETAILED DESCRIPTION OF THE INVENTION

### I. Introduction

The present invention relates to the identification of inhA promoters. In some embodiments, the inhA promoters may be inducible promoters. The inhA promoters can be used for the expression of heterologous genes and recombinant protein production in host cells and particularly in bacterial host cells such as Bacillus and Streptomyces.

### II. The Promoters of this Invention

A promoter region of a B. megaterium inhA gene was identified by bioinformatics analysis and is illustrated in SEQ ID NO: 1, wherein the putative ribosome binding region or site (“RBS”) is indicated by underlining and in bold.

(SEQ ID NO: 1)

GAGCTGGAAAAGAAAGGGAAATCCGTGAGTTATGAAGGGGATTTCTTGTT
TAAAATTGATGAAAAACCTTATGAAATCGCAGCGCGCAATATGTCTATTT
GGAGCATGTCGCTCTATCATATCTTGCTGCAGCGTCAAAAAGAACATTCT
TAAAATTTATTTGTATACAGAGATTATCGCTTCTCGTCTATTGAGAAGTG
ATTTTTTTATCTTTCTGTATCCATAATGGGGAATTTTTTGTTTTTATATA
ATAGTAATACGGAAAAGATAGTTCTAATTCCCTAAAAAAATACACTTCAT
ATATTTTTAGTTTTGGTTATCGATTTGGCATGGAATTTGCATGTGTAAGA
AGGGTTTATCAGACATTTTAATTGTCAGGAAAAGAAAACTAGTTACATAC
TATAAAACTATTTTACATTATCTACTAGAATCAAAAAATTTTAATAATAT
AGTAAAATGTAGTTGACCTTCCTAGTTTTAGAATATAAAATAAATCTTGC
TAATAGTTTGAAAATTCTAAAATGGAGGGGTTTACT

In one embodiment, an inhA promoter will include the nucleic acid sequence of nucleotides 1-523 of SEQ ID NO: 1 and this DNA sequence is referred to as SEQ ID NO: 3 (e.g. starting with 5′ GAGCT . . . ). In some embodiments, an inhA promoter will comprise the nucleic acid sequence of SEQ ID NO: 2 which includes the nucleic acid sequence of SEQ ID NO: 1 and the following nucleotides, GTACA, at the 3′ end of SEQ ID NO: 1.

In some embodiments, an inhA promoter will include additional nucleotides at the 5′ end of the promoter, for example an inhA promoter may include, 3, 4, 5, 6, 7, 8, 9, 10, 11, 12, 15, 18, or more nucleotides. In some embodiments, the inhA promoter will include the nucleotides CCTTCATAT 5′ of nucleotides depicted in SEQ ID NO: 1, SEQ ID NO: 3 or SEQ ID NO: 2.

In some embodiments, the inhA promoter will include truncations (subsequences) from the 5′ end of SEQ ID NO: 1, SEQ ID NO: 3 or SEQ ID NO: 2, for example, in some embodiments the promoter will include about 400 nucleotides, about 375 nucleotides, about 350 nucleotides, about 325 nucleotides, about 300 nucleotides, about 275 nucleotides, about 250 nucleotides, about 225 nucleotides, about 200 nucleotides, about 175 nucleotides, about 150 nucleotides, about 125 nucleotides, about 100 nucleotides, and about 75 nucleotides and still possess promoter activity.

In some embodiments, a truncated inhA promoter will include less than 500 nucleotides, less than 450 nucleotides, less than 400 nucleotides, less than 350 nucleotides, less than 300 nucleotides, less than 250 nucleotides, less than 200 nucleotides and also less than 150 nucleotides of the promoter of SEQ ID NO: 1.

For example, in one embodiment an inhA promoter of the invention will comprise the nucleotide sequence of

(SEQ ID NO: 4)

TTATCGCTTCTCGTCTATTGAGAAGTGATTTTTTTATCTTTCTGTATCCA
TAATGGGGAATTTTTTGTTTTTATATAATAGTAATACGGAAAAGATAGTT
CTAATTCCCTAAAAAAATACACTTCATATATTTTTAGTTTTGGTTATCGA
TTTGGCATGGAATTTGCATGTGTAAGAAGGGTTTATCAGACATTTTAATT
GTCAGGAAAAGAAAACTAGTTACATACTATAAAACTATTTTACATTATCT
ACTAGAATCAAAAAATTTTAATAATATAGTAAAATGTAGTTGACCTTCCT
AGTTTTAGAATATAAAATAAATCTTGCTAATAGTTTGAAAATTCTAAAA.

In another embodiment, the inhA promoter of the invention will comprise the nucleotide sequence of

(SEQ ID NO: 5)

CTAATTCCCTAAAAAAATACACTTCATATATTTTTAGTTTTGGTTATCGA
TTTGGCATGGAATTTGCATGTGTAAGAAGGGTTTATCAGACATTTTAATT
GTCAGGAAAAGAAAACTAGTTACATACTATAAAACTATTTTACATTATCT
ACTAGAATCAAAAAATTTTAATAATATAGTAAAATGTAGTTGACCTTCCT
AGTTTTAGAATATAAAATAAATCTTGCTAATAGTTTGAAAATTCTAAAA.

In another embodiment, the inhA promoter of the invention will comprise the nucleotide sequence of

(SEQ ID NO: 6)

TTTGGCATGGAATTTGCATGTGTAAGAAGGGTTTATCAGACATTTTAATT
GTCAGGAAAAGAAAACTAGTTACATACTATAAAACTATTTTACATTATCT
ACTAGAATCAAAAAATTTTAATAATATAGTAAAATGTAGTTGACCTTCCT
AGTTTTAGAATATAAAATAAATCTTGCTAATAGTTTGAAAATTCTAAAA.

In another embodiment, the inhA promoter of the invention will comprise the nucleotide sequence of

(SEQ ID NO: 7)

ACTATAAAACTATTTTACATTATCTACTAGAATCAAAAAATTTTAATAAT
ATAGTAAAATGTAGTTGACCTTCCTAGTTTTAGAATATAAAATAAATCTT
GCTAATAGTTTGAAAATTCTAAAA.

In other embodiments, a truncated inhA promoter of the invention will comprise the nucleotide sequence illustrated in SEQ ID NO: 23, SEQ ID NO: 24, SEQ ID NO: 25 and SEQ ID NO: 26 and reference is made to Example 4.

It is understood that the promoters of this invention are not limited to SEQ ID NO:1, SEQ ID NO: 3 or SEQ ID NO: 2; rather, the promoters encompass variants and truncations of SEQ ID NO: 3, SEQ ID NO: 4, SEQ ID NO: 5 SEQ ID NO: 6 or SEQ ID NO: 7, SEQ ID NO: 23, SEQ ID NO: 24, SEQ ID NO: 25 and SEQ ID NO: 26 or truncations thereof, and also include promoters having at least 90%, at least 91%, at least 92%, at least 93%, at least 94%, at least 95%, at least 96%, at least 97%, at least 98%, and at least 99% sequence identity to SEQ ID NO: 1, SEQ ID NO: 3, SEQ ID NO: 4, SEQ ID NO: 5, SEQ ID NO: 6; SEQ ID NO: 7, SEQ ID NO: 23, SEQ ID NO: 24, SEQ ID NO: 25; SEQ ID NO: 26; SEQ ID NO: 2 and truncations thereof having promoter activity. In some embodiments, a DNA sequence comprising promoter activity will have at least 95%, 96%, 97%, or 98% sequence identity to the DNA sequence illustrated in SEQ ID NO: 6. In some embodiments, the inhA promoter comprising a DNA sequence having at least 95% sequence identity to SEQ ID NO: 1, SEQ ID NO: 3, SEQ ID NO: 4, SEQ ID NO: 5, SEQ ID NO: 6, SEQ ID NO: 7, SEQ ID NO: 23, SEQ ID NO: 24, SEQ ID NO: 25; SEQ ID NO: 26 or SEQ ID NO: 2 will include one or more (e.g., 1, 2, 3, 4, 5, 6, 7, 8, 9, 10 or more) changes relative to said sequence.

As noted herein, the nucleic acid sequence of SEQ ID NO: 1 (which includes SEQ ID NO: 3) was obtained from Bacillus megaterium. As would be readily apparent to one of skill in the art, variants of SEQ ID NO: 1 or SEQ ID NO: 3 and truncated sequences thereof that retain promoter activity can be identified by identifying sequences that are similar to SEQ ID NO: 1 or SEQ ID NO: 3 in other bacterial species and particularly other Bacillus strains.

In some embodiments, an inhA promoter of the invention will be one that hybridizes under high or very high stringency conditions to the sequence of SEQ ID NO: 3 or a truncated sequence thereof “Hybridization conditions” refer to the degree of “stringency” of the conditions under which hybridization is measure. Hybridization conditions can be based in the melting temperature (T_{M}) of the nucleic acid binding complex, as taught by Berger and Kimmel (1987, Guide to Molecular Cloning techniques, Methods in Enzymology, Vol. 152, Academic Press, San Diego, Calif.). High stringency conditions typically occurs at about 5° C. to 10° C. below the Tm. Hybridization conditions may also be based on washing conditions employed after hybridization as known in the art. For example, high stringency conditions refer to washing with a solution of 0.2×SSC/0.1% SDS at 37° C. for 45 minutes.

In some embodiments, an inhA promoter will be a variant inhA promoter. Standard library construction methods which are well known in the art can be used for the generation of promoter variants. For example, mutagenesis and directed evolution methods can be readily applied to polynucleotides (such as, for example, the wild-type inha promoter sequence (e.g., SEQ ID NO: 1). See specifically, e.g., Ling, et al., “Approaches to DNA mutagenesis: an overview,” Anal. Biochem., 254(2):157-78 (1997); Hemsley et al., “A simple method for site-directed mutagenesis using the polymerase chain reaction.” Nucleic Acids Res. 17(16): 6545-51 (1989); and Matsmura, et al., “Optimization of heterologous gene expression for in vitro evolution.” Biotechniques 30(3): 474-6 (2001). Other general references include the following Dale, et al., “Oligonucleotide-directed random mutagenesis using the phosphorothioate method,” Methods Mol. Biol., 57:369-74 (1996); Smith, “In vitro mutagenesis,” Ann. Rev. Genet., 19:423-462 (1985); Botstein, et al., “Strategies and applications of in vitro mutagenesis,” Science, 229:1193-1201 (1985); Carter, “Site-directed mutagenesis,” Biochem. J., 237:1-7 (1986); Kramer, et al., “Point Mismatch Repair,” Cell, 38:879-887 (1984); Wells, et al., “Cassette mutagenesis: an efficient method for generation of multiple mutations at defined sites,” Gene, 34:315-323 (1985); Minshull, et al., “Protein evolution by molecular breeding,” Current Opinion in Chemical Biology, 3:284-290 (1999); Christians, et al., “Directed evolution of thymidine kinase for AZT phosphorylation using DNA family shuffling,” Nature Biotechnology, 17:259-264 (1999); Crameri, et al., “DNA shuffling of a family of genes from diverse species accelerates directed evolution,” Nature, 391:288-291; Crameri, et al., “Molecular evolution of an arsenate detoxification pathway by DNA shuffling,” Nature_Biotechnology, 15:436-438 (1997); Zhang, et al., “Directed evolution of an effective fucosidase from a galactosidase by DNA shuffling and screening,” Proceedings of the National Academy of Sciences, U.S.A., 94:45-4-4509; Crameri, et al., “Improved green fluorescent protein by molecular evolution using DNA shuffling,” Nature Biotechnology, 14:315-319 (1996); Stemmer, “Rapid evolution of a protein in vitro by DNA shuffling,” Nature, 370:389-391 (1994); Stemmer, “DNA shuffling by random fragmentation and reassembly: In vitro recombination for molecular evolution,” Proceedings of the National Academy of Sciences, U.S.A., 91:10747-10751 (1994); WO 95/22625; WO 97/0078; WO 97/35966; WO 98/27230; WO 00/42651; and WO 01/75767.

In some embodiments, the promoter encompassed by the invention is a variant promoter or a truncated variant promoter comprising a nucleotide sequence of any one of the sequences selected from SEQ ID NOs: 27-63 (e.g. those variants listed in Table 5) when said sequence is aligned with the ribosome binding site of SEQ ID NO: 1 or a truncated sequence thereof, for example but not limited to SEQ ID NO: 23. In some embodiments, a variant inhA promoter or a truncated variant inhA promoter will include 1, 2, 3, 4, 5, 6, 7, 8, 9, or 10 different polynucleotides as compared to the inhA promoter of SEQ ID NO: 1 or SEQ ID NO: 3. In some embodiments, a variant inhA promoter will include substitutions, additions and/or deletions in the −35 to −10 region of the inhA promoter depicted in SEQ ID NO: 1, SEQ ID NO: 3 or SEQ ID NO: 2. In some embodiments, the variant will include 1, 2, 3, 4 or 5 substitutions, and/or additions in the −35 to −10 region. In some embodiments, a variant promoter of the invention includes a substitution, addition or deletion (e.g., 1, 2, 3, 4, 5, 6, 7, 8, 9 or 10 substitutions, additions or deletions) between the start codon (ATG) and the RBS (e.g. TGGAGGGG). In some preferred embodiments, a variant promoter of the invention will include between 1 and 5 additions, substitutions and/or deletions in the region between the start codon and the RBS. In some embodiments, a variant promoter of the invention includes a substitution, addition or deletion (e.g., 1, 2, 3, 4, 5, 6, 7, 8, 9 or 10 substitutions, additions or deletions) between −10 region and the RBS (e.g. TGGAGGGG) when compared with the promoter sequence of SEQ ID NO: 1 or a truncated sequence thereof for example but not limited to SEQ ID NO: 23.

In some embodiments, a variant, a truncated variant or a truncated inhA promoter will have at least the same level of promoter activity as the activity exhibited by SEQ ID NO: 1, SEQ ID NO: 3 or SEQ ID NO: 2. In some embodiments, the promoter activity will be greater, for example at least 5%, at least 10%, at least 15%, at least 20%, at least 25%, at least 30%, at least 40% and at least 50% greater promoter activity as compared to the activity exhibited by SEQ ID NO: 1, SEQ ID NO: 3 or SEQ ID NO: 2. In some embodiments, the promoter activity will be greater, for example at least 5%, at least 10%, at least 15%, at least 20%, at least 25%, at least 30%, at least 40% and at least 50% greater promoter activity as compared to the activity exhibited by SEQ ID NO: 1, SEQ ID NO: 3 or SEQ ID NO: 2.

In some preferred embodiments, the inhA promoters of the invention comprise the same ribosome binding region (RBS) as set forth in SEQ ID NO:2 (e.g., an inhA promoter of SEQ ID NO: 23, SEQ ID NO: 24, SEQ ID NO: 25, SEQ ID NO: 26 or a sequence having at least 97% sequence identity thereto). In some embodiments, the RBS may be different than the RBS of SEQ ID NO: 2.

### III. Constructing Expression Vectors

To produce a vector such as an expression cassette utilizing the inhA promoters of this invention for gene expression, a variety of methods well known in the art may be used to first obtain the polynucleotide sequences for the promoter and the coding sequence of interest, and subsequently join the two sequences so that they are operably linked for gene expression. The coding sequence is heterologous to the promoter. Typically, the coding sequence is from a source different from the source of the promoter.

In some embodiments, the coding sequence encodes for a protein such as an enzyme, a therapeutic protein, a reporter protein, a receptor protein and the like. In some preferred embodiments, the protein is an enzyme. The enzyme may be a cellulase, a hemicellulase, a glucoamylase, an amylase (e.g. an alpha amylase or a beta amylase), a protease (e.g., an acid protease, an alkali protease, a neutral protease, pepsin, peptidase, trypsin, chymosin or subtilisin), a phytase, a lipase, an esterase, a xylanase, an oxidoreductase, a laccase, a cutinase, an isomerase (e.g., a glucose isomerase or a xylose isomerase), a pullulanase, a phenol oxidizing enzyme, a starch hydrolyzing enzyme, a mannase, a catalase, a glucose oxidase, a transferase, a lyase (e.g., pectate lyase or acetolactate decarboxylase). These enzymes may originate from bacteria or fungi. In addition, the protein or enzyme may be a variant of a wild type or naturally occurring enzyme.

In some particular embodiments, the enzyme is a cellulase. Cellulases are enzymes that hydrolyze the β-D-glucosidic linkages in cellulose. Cellulolytic enzymes are generally divided into three classes of enzyme activities and these enzymes work in coordinated fashion with each other to break down cellulose. The endoglucanases (E.C. 3.2.1.4 also called β-1,4 endoglucanases) cleave β 1,4 glycosidic linkages randomly along the cellulose chain. Exoglucanases (E.C. 3.2.1.91, also called cellobiohydrolases (CBH)) cleave cellobiose from either the reducing or non-reducing end of a cellulose chain. β-glucosidases (E.C. 3.2.1.21 also called cellobioses) hydrolyze aryl and alkyl-β-D-glucosides. Numerous cellulases are known and described in the literature, for example cellulases described in U.S. Pat. No. 6,287,839; U.S. Pat. No. 6,562,612; Jung et al. (1993) Appl. Environ. Microbiol. 59:3032-3043 and Lao et al, (1991) J. Bacteriol. 173:3397-3407. In two of the specific examples of this disclosure an inhA promoter is operably linked to a heterologous polynucleotide encoding a native β-glucosidase referred to as CelA and also an inhA promoter is operably linked to a heterologous polynucleotide encoding a native exocellulase referred to as CbhII.

In some particular embodiments, the enzyme is a protease, such as a serine, metallo, thiol or acid protease. In some embodiments, the enzyme will be a serine protease, such as but not limited, to subtilisin.

In some embodiments, the coding sequence which is operably linked to the inhA promoter of the invention will be a protein other than an enzyme, for example the protein may include, hormones (e.g., follicle-stimulating hormone, erythropoietin, insulin and the like), receptors, growth factors (e.g., epidermal growth factors, cytokines, fibroblast growth factors and the like), antigens and antibodies (e.g., class G, A, M, or E classes).

In some embodiments, the protein may be a reported protein such as but not limited to β-galatosidase (lacZ), β-glucuronidase (GUS), fluorescent protein (GFP), chloramphenicol, and acetyl transferase (CAT).

In some embodiments, especially when the coding sequence codes for an extracellular protein, a signal sequence will be linked to the N-terminal portion of the coding sequence to facilitate the secretion of the protein. The signal sequence may be endogenous or exogenous to the host organism and further the signal sequence may be one naturally associated with the coding sequence or heterologous to the coding sequence. In some embodiments, the signal sequence comprises a Bacillus signal sequence such as those derived from (e.g., B. stearothermophilus, B. licheniformis, B. subtilis or B. megaterium). In some embodiments, the signal sequence will be derived from a Bacillus megaterium strain. (Malten et al., (2005) Biotechnol. Bioeng. 91:616-621). A foreign signal peptide coding region may be required where the coding sequence does not normally contain a signal peptide coding region. Alternatively, the foreign signal peptide coding region may simply replace the natural signal peptide coding region in order to obtain enhanced secretion of the enzyme relative to the natural signal peptide coding region normally associated with the coding sequence.

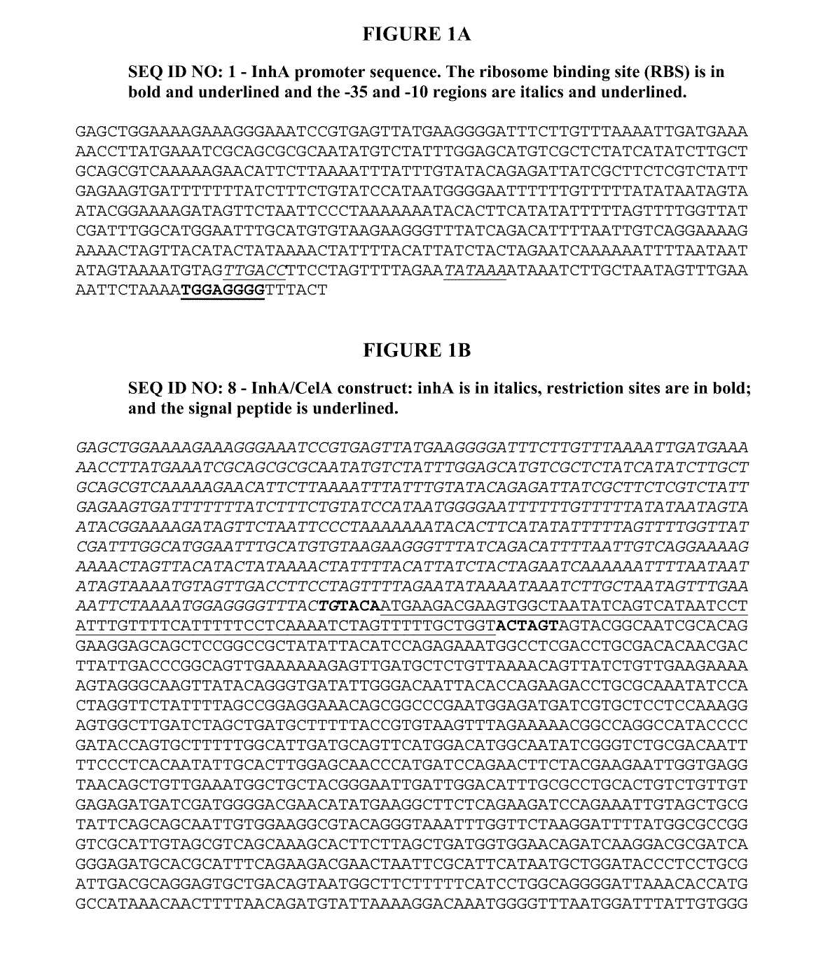

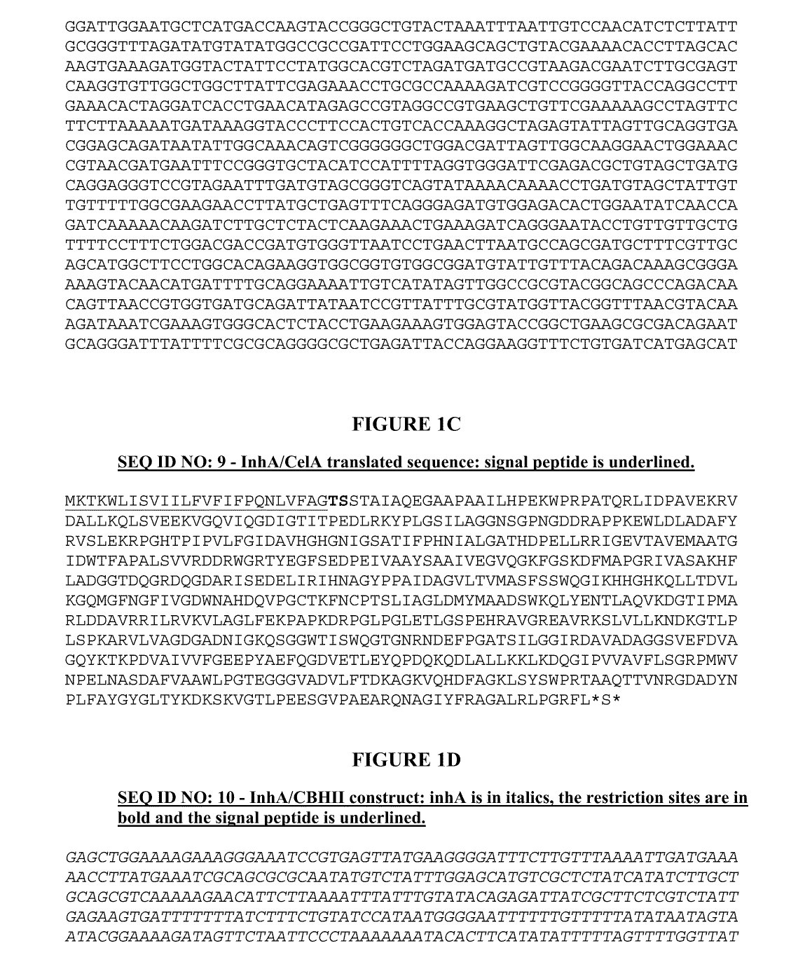

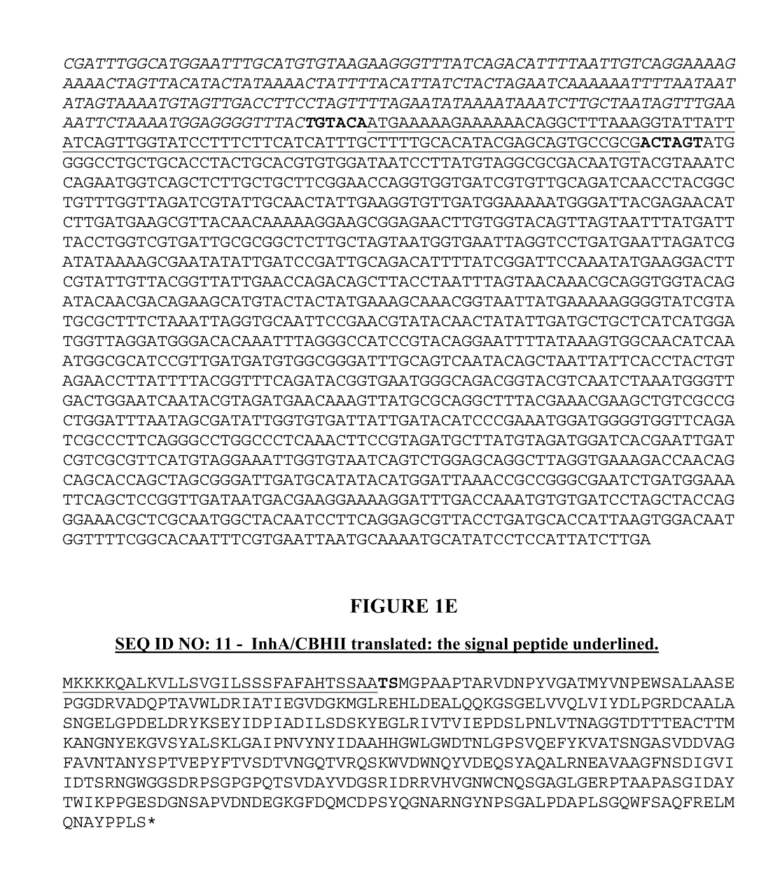

In some embodiments, the signal sequence will be a signal sequence of a cellulase coding gene. In some embodiments, the signal sequence will be a signal sequence of a protease coding gene such as a neutral protease or a serine protease. In some embodiments, the signal sequence will have the sequence as illustrated by the signal sequences illustrated in FIG. 1 and incorporated into SEQ ID NO: 8 and SEQ ID NO: 10.

Additionally, effective signal peptide sequences may be obtained from the genes for Bacillus NClB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, Microbiol Rev. 57: 109-137 (1993).

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. However, any signal peptide coding region capable of directing the expressed polypeptide into the secretory pathway of a host cell of choice may be used in the present invention and one skilled in the art is well aware of numerous signal sequences that may be used depending on the protein being produced and secreted in a host organism.

Many polynucleotide sequences can be cloned from a source organism or genomic or cDNA library, using conventional methods such as polymerase chain reaction (PCR)-based cloning methodology. Alternatively, they can be obtained by chemical synthesis. Methods of chemically synthesizing polynucleotide sequences are well known in the art, e.g., the solid phase phosphoramidite triester method first described by Beaucage and Caruthers, Tetrahedron Lett. 22: 1859-1862 (1981), using an automated synthesizer, as described in Van Devanter et. al., Nucleic Acids Res. 12: 6159-6168 (1984). Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

Purification of polynucleotide sequences is performed using any art-recognized strategy, e.g., native acrylamide gel electrophoresis or anion-exchange HPLC as described in Pearson and Reanier, J. Chrom. 255: 137-149 (1983). All cloned or synthetic polynucleotide sequences can be verified using, e.g., the chain termination method for sequencing double-stranded templates of Wallace et al., Gene 16: 21-26 (1981).

Once the promoter sequence and coding sequence are obtained and verified, they can then be ligated to produce a vector (expression cassette) in which the promoter and the heterologous coding sequence are operably linked. A number of known methods are suitable for the purpose of ligating the two sequences, such as ligation methods based on PCR and ligation methods mediated by various ligases (e.g., bacteriophase T4 ligase). The promoter used to direct expression of a heterologous sequence is optionally positioned about the same distance from the heterologous transcription start site as it is from the transcription start site in its natural setting. As is known in the art, however, some variation in this distance can be accommodated without loss of promoter function.

To obtain high level expression of a heterologous protein by the expression cassette of the present invention, one may also include in the expression cassette a transcription/translation terminator and a ribosome binding site for translational initiation. In addition to the promoter, the expression cassette optionally contains all the additional elements required for the expression of the heterologous sequence in host cells, such as signals required for efficient polyadenylation of the transcript, ribosome binding sites, translation termination, enhancers, and a cleavable signal peptide sequence to promote secretion of the polypeptide by the transformed cells. If genomic DNA is used as the heterologous coding sequence, introns with functional splice donor and acceptor sites may also be included.

The elements that are typically included in expression vectors also include a replicon that functions in some bacterial cells, a gene encoding antibiotic resistance to permit selection of microorganisms that harbor recombinant plasmids, and unique restriction sites in nonessential regions of the plasmid to allow insertion of heterologous sequences. The particular antibiotic resistance gene chosen is not critical, as any of the many resistance genes known in the art are suitable. Similar to antibiotic resistance selection markers, metabolic selection markers based on known metabolic pathways may also be used as a means for selecting transformed host cells.

The particular vector used to transport the expression cassette into the cell is not particularly critical. Any of the conventional vectors used for expression in prokaryotic or eukaryotic cells may be used. Standard bacterial expression vectors include plasmids such as pBR322 based plasmids, pSKF, pET23D, and fusion expression systems such as GST and LacZ. Epitope or affinity tags can be added to recombinant proteins to provide convenient methods of isolation, e.g., c-myc, HIS-6, MBP, and FLAG.

### IV. Transformation and Recombinant Gene Expression

In some embodiments, a transformed host cell is a eukaryotic cell. Suitable eukaryotic host cells include, but are not limited to, fungal cells, algal cells, insect cells, and plant cells.

Suitable fungal host cells include yeast cells and filamentous fungal cells. Some preferred filamentous fungal host cells include but are not limited Aspergillus, Chrysosporium, Corynascus, Fusarium, Humicola, Hypocrea, Myceliophthora, Mucor, Neurospora, Penicillium, Rhizomucor, Rhizopus, Talaromyces, Thermoascus, Thielavia, Trametes, Trichoderma, or teleomorphs, or anamorphs, and synonyms or taxonomic equivalents thereof.

In some embodiments of the invention, the filamentous fungal host cell is of the Trichoderma species, e.g., T. longibrachiatum, T. viride (e.g., ATCC 32098 and 32086), Hypocrea jecorina or T. reesei (NRRL 15709, ATTC 13631, 56764, 56765, 56466, 56767 and RL-P37 and derivatives thereof—See Sheir-Neiss et al., Appl. Microbiol. Biotechnology, 20 (1984) pp 46-53), In addition, the term “Trichoderma” refers to any fungal strain that was previously classified as Trichoderma or currently classified as Trichoderma. In some embodiments of the invention, the filamentous fungal host cell is of the Aspergillus species, e.g., A. awamori, A. fumigatus, A. japonicus, A. nidulans, A. niger, A. aculeatus, A. foetidus, A. oryzae, A. sojae, and A. kawachi. (Reference is made to Kelly and Hynes (1985) EMBO J. 4,475479; NRRL 3112, ATCC 11490, 22342, 44733, and 14331; Yelton M., et al., (1984) Proc. Natl. Acad. Sci. USA, 81, 1470-1474; Tilburn et al., (1982) Gene 26, 205-221; and Johnston, I. L. et al. (1985) EMBO J. 4, 1307-1311). In some embodiments of the invention, the filamentous fungal host cell is of the Chrysosporium species, e.g., C. lucknowense. In some embodiments of the invention, the filamentous fungal host cell is of the Fusarium species, e.g., F. bactridioides, F. cerealis, F. crookwellense, F. culmorum, F. graminearum, F. graminum, F. oxysporum, F. roseum, and F. venenatum. In some embodiments of the invention, the filamentous fungal host cell is of the Myceliophthora species, e.g., M. thermophilic. In some embodiments of the invention, the filamentous fungal host cell is of the Neurospora species, e.g., N. crassa. Reference is made to Case, M. E. et al., (1979) Proc. Natl. Acad. Sci. USA, 76, 5259-5263; U.S. Pat. No. 4,486,553; and Kinsey, J. A. and J. A. Rambosek (1984) Molecular and Cellular Biology 4, 117-122. In some embodiments of the invention, the filamentous fungal host cell is of the Humicola species, e.g., H. insolens, H. grisea, and H. lanuginosa. In some embodiments of the invention, the filamentous fungal host cell is of the Penicillum species, e.g., P. purpurogenum, P. chrysogenum, and P. verruculosum. In some embodiments of the invention, the filamentous fungal host cell is of the Thielavia species, e.g., T. terrestris. In some embodiments of the invention, the filamentous fungal host cell is of the Trametes species, e.g., T. villosa and T. versicolor.

In the present invention a yeast host cell may be a cell of a species of, but not limited to Candida, Hansenula, Saccharomyces, Schizosaccharomyces, Pichia, Kluyveromyces, and Yarrowia.

In some embodiments on the invention, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative and gram-variable bacterial cells. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, and Zymomonas. In yet other embodiments, the bacterial host strain is non-pathogenic to humans. In some embodiments the bacterial host strain is an industrial strain. Numerous bacterial industrial strains are known and suitable in the present invention.

In some embodiments, of the invention the bacterial host cell is of the Bacillus species, e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. Some preferred embodiments of a Bacillus host cell include B. subtilis, B. licheniformis, B. megaterium, B. stearothermophilus and B. amyloliquefaciens. In some embodiments, the Bacillus strain will be a deletion strain, for example a protease deletion strain such as an exoprotease deficient strain (Wittch et al., (1995) Appl. Microbiol. Biotech 42:871-877 and Malten et al., (2006) Appl. Environ. Microbiol. 72:1677-1679). In some embodiments, the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans. In some embodiments the bacterial host cell is of the Zymomonas species, e.g., Z. mobilis, and Z. lipolytica.

Strains which may be used in the practice of the invention including both prokaryotic and eukaryotic strains are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). Specific examples of heterologous expression of cellulase genes can be found in Bacillus, E. coli, Streptomyces, and Trichoderma (See, for example Lejeune et al Biosynthesis and Biodegradation of Cellulose; Haigler, C. et al Marcel Dekker, NY, N.Y. 1990 pg 623-671).

Standard transformation methods may be used to produce recombinant host cells harboring the expression vector of the invention. For example, introduction of a vector into a host cell can be effected by calcium phosphate transfection, DEAE-Dextran mediated transfection, electroporation, PEG-mediated protoplast transformation or other common techniques (See Davis, L., Dibner, M. and Battey, I. (1986) Basic Methods in Molecular Biology, Ausubel et al., 1994, Current Protocols in Molecular Biology; Campbell et al., 1989, Curr Genet. 16:53-56; U.S. Pat. No. 6,255,115; U.S. Pat. No. 5,264,366, U.S. Pat. No. 5,364,770; U.S. Pat. No. 6,022,725; Brigid, et al., 1990 FEMS Microbiol Lett. 55: 135-138 and Hopwood et al., Genetic Manipulation of Streptomyces: A Laboratory Manual (1985) John Innes Foundation, Norwich, UK)).

Production of the heterologous protein by the transformed host cells can be detected and confirmed by any immunoassays known in the art, e.g., ELISA. Upon confirmation, the protein can then be purified by standard techniques (see, e.g., Colley et al., J. Biol. Chem. 264: 17619-17622 (1989); Guide to Protein Purification, in Methods in Enzymology, vol. 182 (Deutscher, ed., 1990)).

In addition to plasmid-based DNA construction, the DNA construct or sequence comprising an inhA promoter and a heterologous coding sequence may be chromosomally integrated into the host genome such as by homologous recombination. Chromosomal integration may result in certain advantages over plasmid-based constructions.

The recombinant or engineered host cells can be cultured in conventional nutrient media modified as appropriate for activating promoters and selecting transformants. Culture conditions, such as temperature, pH and the like will be apparent to those skilled in the art and in the references cited herein, including, for example, Sambrook, Ausubel and Berger, as well as, for example, Freshney (1994) Culture of Animal Cells, a Manual of Basic Technique, third edition, Wiley-Liss, New York; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla., all of which are incorporated herein by reference. Some preferred culture conditions may be found in Harwood et al. (1990) Molecular Biological Methods For Bacillus, John Wiley and/or from the American Type Culture Collection (ATCC).

In some embodiments, cells comprising a inhA promoter and expressing a coding sequence are grown under batch, fed-batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady sate growth conditions. Methods for modulating nutrients and growth factors for continuous fermentation processes as well as techniques for maximizing the rate of product formation are well known in the art of industrial microbiology.

In some embodiments of the invention, it will be desirable for the inhA promoter to be active under low carbon-substrate conditions. Carbon-substrates used during the culturing of recombinant host cells may include but are not limited to organic acids (such as, but not limited to acetate, succinate, propionate, benzoate, hydroxybenzoate, pyruvate and gluconate), monosaccharide and disaccharide sugars (such as, but not limited to glucose, galactose, fructose, mannose, ribose, rhamnose, sorbose, xylose, lactose, trehalose, saccharose, maltose and sucrose), alcohols (such as, but not limited to inositol, methanol, ethanol, and glycerol, starch, cellobiose, molasses, and the like). In particular, the carbon-source includes a C5 or C6 fermentable sugar and particularly a C6 sugar, such as glucose. For example, in a fed-batch fermentation system the level of carbon-substrate and particularly glucose may be maintained at a relatively low concentration. In some systems the level of glucose will be kept at less than 1.0 g/L, less than 0.25 g/L, less than 0.10 g/L, less than 0.05 g/L and also less than 0.025 g/L of media. In some embodiments, the inhA promoters of the invention will be active under these fermentation conditions and further may be active without the addition of exogenous inducing agents such as a chemical inducer, e.g., IPTG.

In some embodiments, the invention encompasses a process for producing a protein in a microbial host cell wherein the host cell is a recombinant bacterial cell (e.g., a Bacillus cell or E. coli) which comprises transforming the microbial host cell with a vector comprising an inhA promoter encompassed by the invention operably linked to a polynucleotide coding for an enzyme of interest and culturing the transformed microbial cell under culture conditions suitable for the expression and production of the enzyme. In some preferred embodiments, the enzyme is a cellulase, a protease, an amylase or a xylanases enzyme and the host cell is a bacterial cell. In some embodiments, the enzyme that is produced by the process is further isolated from the culture media.

### EXAMPLES

The following examples are provided by way of illustration only and not by way of limitation. Those of skill in the art will readily recognize a variety of non-critical parameters that could be changed or modified to yield essentially the same or similar results.

### Example 1

### Isolation and Sequencing of an inhA Promoter from B. megaterium Chromosomal DNA

The inhA protein is an extracellular metalloprotease whose expression is induced during late exponential or stationary phase (Eur J Biochem; 1984, 139, 247-252) (Microbiology; 2001, 147, 1805-1813). The inhA promoter was amplified from the chromosomal DNA of B. megaterium and tested against two different genes coding for cellulase enzymes. One gene was a β-glucosidase gene obtained from Azospirillum irakense and designated celA and the other was an exoglucanase gene obtained from Streptomyces sp and designated cbhII.

### Genomic DNA Prep:

To isolate chromosomal DNA from B. megaterium, 3 ml of overnight cell culture in Luria Broth (LB) was harvested at 5000 g for 5 min at room temperature and resuspended in 100 ul of SMM buffer (0.5M Sucrose, 20 mM Maleate, 20 mM MgCl₂, pH 6.5). Lysozyme (chicken egg white, Sigma) solution was added to a final concentration of 1 mg/ml and cells were incubated at 42° C. for 30 min. After the incubation, cells were washed once with SMM buffer and resuspended in 100 μl of water. The genomic DNA was extracted from the lysozyme treated cells by repeated phenol-chloroform extraction and precipitation with 70% isopropanol. Precipitated DNA pellet was re-suspended in 100 μl of warm water and used as the template DNA.

### InhA Promoter Extraction and Confirmation:

The native inhA promoter was amplified from the wildtype (WT) B. megaterium genomic DNA by PCR amplifying a 500 base pair region upstream of the native inhA gene using primers P1 and P2 (Table 1);

TABLE 1

| Name | | | |
|---|---|---|---|
| of the | | Restriction | SEQ ID |
| primer | Sequence (5′ → 3′) | site | NO: |
| P1 | ccttcatatgagctggaaaagaaagggaaatccgtgag | NdeI | 12 |
| P2 | ggtatgtacagtaaacccctccattttagaattttcaaac | BsrG1 | 13 |
| P3 | tactgtacaatgaaaaagaaaaaacaggctttaaaggta | BsrG1 | 14 |
| P4 | ccatactagtcgcggcactgctcgtatgtgcaaaagcaaa | SpeI | 15 |
| P5 | ttactgtacaatgaagacgaagtggctaatatcagtcata | BsrG1 | 16 |
| P6 | tactactagtaccagcaaaaactagattttgaggaaaaat | SpeI | 17 |

The Clontech Advantage HF 2 PCR kit containing Advantage 2 enzyme blend of TITANIUM™ Taq DNA Polymerase and a small amount of proofreading enzyme was used to PCR amplify the inhA promoter. PCR was done in 50 μul volume as follows.

Advantage Buffer: (5 μl); dNTPs: (5 μl); 20 μM Fwd primer: (1 μl); 20 μM Rev primer: (1 μl);

### Polymerase: (1 μl); Genomic DNA: (0.5 μl); and H₂O: (36.5 μl).

PCR conditions: 94° C.-2 min; 28 cycles of (94° C.-30 sec, 55° C.-30 sec, 72° C.-30 sec); 72° C.-10 min.

PCR product was purified using standard Qiagen PCR purification protocol supplied by the manufacturer and digested overnight with NEB restriction enzymes NdeI and BsrG1 in NEB buffer 4 and BSA. PCR product was cloned into the Nde1/BsrG1 digested shuttle vector pBmX-e (pMM1522-Boca Scientific, Boca Raton, Fla.), a shuttle vector that replicates in both B. megaterium and E. coli. The inhA construct was sequence confirmed from this newly constructed vector pBmI-e (Table 2).

### Example 2

### Construction of Plasmids Comprising the Promoters, Signal Peptides and Genes

### Plasmid Construction:

Two plasmids were constructed. Plasmid BmI-PG-celA comprising the inhA promoter, signal peptide (PenG) and heterologous gene celA (SEQ ID NO: 8) and plasmid pBmI-NM-cbhII comprising the inhA promoter, signal peptide (NprM) and heterologous cbhII (SEQ ID NO: 10) and used to determine the activity and the strength of the inhA promoter.

Vectors pBmX-PG-celA and pBmX-NM-cbhII were previously synthesized by first cloning signal peptides at the BsrG1 and SpeI sites of pBmX-e vector and followed by cloning genes (celA or cbhII) at the SpeI and NgoMIV sites. These vectors were further used as base vectors to build the desired plasmids for inhA promoter studies. Signal peptides NprM and PenG were obtained from the WT B. megaterium (DSM319) genomic DNA by PCR amplification using primers (P3, P4) and (P5, P6) respectively (Table 1). PCR amplification method was used as described in Example 1.

The celA and cbhII gene sequences were obtained from the genome sequences of Azospirillum irakense and Streptomyces sp. respectively, codon optimized for Bacillus and synthesized by GenScript, Piscataway, N.J.

To build the inhA/cbhII construct, the pBmI-e vector containing inhA was digested with NdeI/BsrG1, the resulting fragment containing the inhA promoter was cloned into pBmX-NM-cbhII vector at the NdeI and BsrG1 site replacing the xylA promoter. The resulting plasmid pBmI-NM-cbhII included the NprM-cbhII gene downstream of the inhA promoter.

TABLE 2
Various vectors used in the examples

| Name of the vector | promoter | Signal Peptide | Gene |
|---|---|---|---|
| pBmX-e | xylose | None | None |
| pBmI-e | inhA | None | None |
| pBmX-PG-celA | xylose | PenG | celA |
| pBmI-PG-celA | inhA (GPI) | PenG | celA |
| pBmX-NM-cbhII | xylose | NprM | cbhII |
| pBmI-NM-cbhII | inhA (GPI) | NprM | cbhII |

To build the inhA/celA construct, the pBmX-PG-celA vector was digested with BsrGI/NgoMIV and the resulting fragment containing PG signal peptide coding region and celA was litigated into the pBmI-e vector digested with BsrG1 and NgoMIV. The resulting vector pBmI-PG-celA included the PenG-celA gene downstream of the inhA promoter. Generally known ligation protocols were followed for introducing vectors into Bacillus.

### Example 3

Transformation and Assay

### Protoplast Preparation and Transformation:

Protoplast preparation of a B. megaterium strain derived from WH320 containing protease deletions (ΔnprM, Δspo0A, ΔspoIVB) and transformations were carried out as described by Brown and Carlton (J. Bacteriology, (1980) 142(2): 508-512). Since ligation mixes were transformed directly into the B. megaterium strains, transformants containing celA constructs were established by growing 10 to 15 colonies separately in 10 ml of semi-defined A5 media (Yang et al., (2006) Microb. Cell Fact. 5: 36) in shake flasks for 16 hrs at 37° C. and 220 RPM, followed by a series of steps outlined below.

CelA expression and secretion was measured by monitoring its pNPG hydrolysis activity. (Faure et al., (1999) J. Bacteriol. 181:3003-3009) 20 μl of shake flask supernatant sample was added to 80 ul of pNPG solution (4 mM pNPG 50 mM potassium phosphate buffer pH 7) in a 96 well microtiter plate. The plate was incubated at 50° C. for 1 hr and scanned at 405 nm in SpectraMax High-Throughput Microplate Spectrophotometer. Positive clones were ascertained to be the samples with absorbance three times that of the background. These were further confirmed by sequencing and subsequently analyzed in fermenters.

Transformations of the cbhII constructs were verified by growing 6 to 8 colonies separately in 10 ml of semi-defined A5 media for 16 hrs at 37° C. Protein production and secretion from each sample was confirmed by running the shake flask supernatant sample on SDS-PAGE. 30 ul samples were loaded onto the Invitrogen 1.5 mm SDS-gel and run as per manufacturer instructions. Positive samples were identified as those that displayed a visible band at 45 kD. These clones were further confirmed by sequencing and analyzed in fermenters.

### Assay:

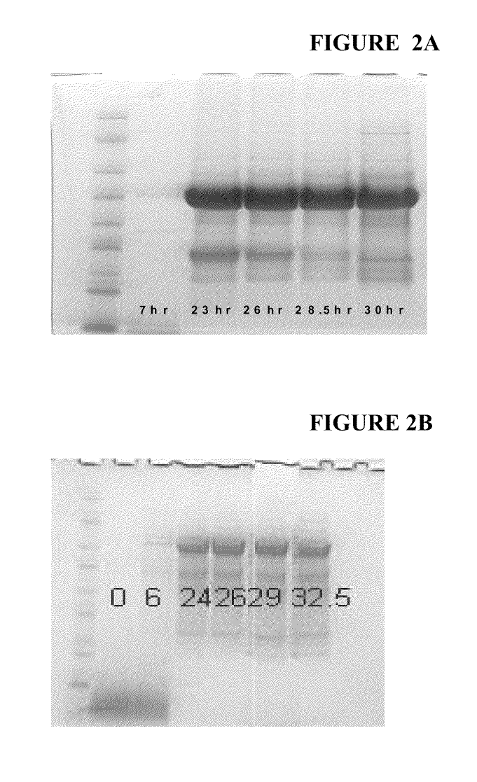

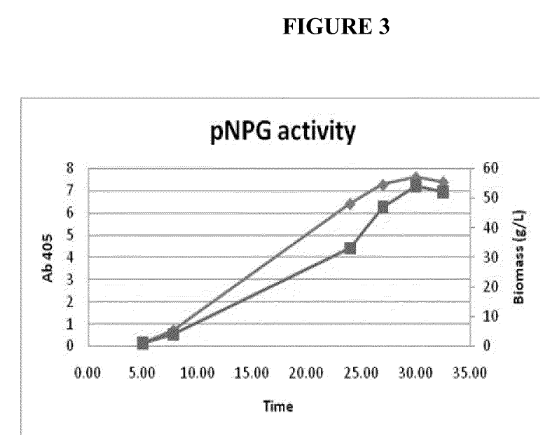

The inhA promoter constructs as described above (pBmI-PG-celA and pBmI-NM-cbhII) were tested in 2 L fermenters. Time points were taken and supernatant samples from the fermenters were run on SDS-PAGE. Based upon gel analysis (FIG. 2) cells secreted heterologous protein with the inhA promoter in 2 L fermentations. In addition FIG. 3 illustrates the secretion of CelA protein as measured by pNPG hydrolysis.

### Example 4

### Promoter Truncations

Four truncated promoters were constructed using the BmI-PG-celA plasmid described in Example 1. The plasmids, designated BmI386, BmI301, BmI251 and BmI201 comprised inhA promoter sequences SEQ ID NO: 26, SEQ ID NO: 25, SEQ ID NO: 24 and SEQ ID NO: 23, respectively. The promoters were constructed using the reverse primer and the relevant forward primer listed in Table 3 below.

The primers were first phosphorylated at 37° C. for 2 hrs as follows: (100 uM) primer, 10 μl; T4 Lizase Buffer, 5 μl; T4 PNK, 1 μl; H₂O, 34 μl. Phosphorylated primers were then used to amplify the plasmid with the following protocol: Forward primer, 1 μl; inhA_rev, 1 μl; BmI-PG-celA, 1 μl; 10× Herculase Buffer, 5 μl; dNTP's (100 mN), 0.4 μl; DSMO, 1 μl; Herculase Polymerase, 0.5 μl; and H₂O, 40.1 μl. PCR conditions: 95° C.-4 min; 32 cycles of (95° C.-30 sec, 55° C.-40 sec, 72° C.-10 sec); 72° C.-5 min.

The PCR products were DpnI digested for 1 hr at 37° C. and then purified using standard Qiagen PCR purification methods. The purified products were ligated for 4 hrs at 16° C. using Promega's T4 ligase. Ligation mixtures were directly transformed into B. megaterium strains and the constructs were sequence verified.

TABLE 3

| Name | | |
|---|---|---|
| of the | | SEQ ID |
| primer | Sequence (5′ → 3′) | NO: |
| 201_frwd | aattaacatatgtttgcatgtgtaagaagggtttat | 18 |
| 251_frwd | aattaacatatgaaaaatacacttcatatattttta | 19 |
| 301_frwd | aattaacatatgttttgtttttatataatagtaata | 20 |
| 386_frwd | aattaacatatgtaaaatttatttgtatacagagat | 21 |
| inhA_rev | Catatgacgatagattttaatggacccaa | 22 |

The sequences of the truncated promoter corresponding to SEQ ID NOs: 23, 24, 25 and 26 are illustrated below, wherein the RBS is in bold and underlined:

(SEQ ID NO: 23)

TTTGCATGTGTAAGAAGGGTTTATCAGACATTTTAATTGTCAGGAAAAGA
AAACTAGTTACATACTATAAAACTATTTTACATTATCTACTAGAATCAAA
AAATTTTAATAATATAGTAAAATGTAGTTGACCTTCCTAGTTTTAGAATA
TAAAATAAATCTTGCTAATAGTTTGAAAATTCTAAAATGGAGGGGTTTAC
T;

(SEQ ID NO: 24)

AAAAATACACTTCATATATTTTTAGTTTTGGTTATCGATTTGGCATGGAA
TTTGCATGTGTAAGAAGGGTTTATCAGACATTTTAATTGTCAGGAAAAGA
AAACTAGTTACATACTATAAAACTATTTTACATTATCTACTAGAATCAAA
AAATTTTAATAATATAGTAAAATGTAGTTGACCTTCCTAGTTTTAGAATA
TAAAATAAATCTTGCTAATAGTTTGAAAATTCTAAAATGGAGGGGTTTAC
T;

(SEQ ID NO: 25)

TTTTGTTTTTATATAATAGTAATACGGAAAAGATAGTTCTAATTCCCTAA
AAAAATACACTTCATATATTTTTAGTTTTGGTTATCGATTTGGCATGGAA
TTTGCATGTGTAAGAAGGGTTTATCAGACATTTTAATTGTCAGGAAAAGA
AAACTAGTTACATACTATAAAACTATTTTACATTATCTACTAGAATCAAA
AAATTTTAATAATATAGTAAAATGTAGTTGACCTTCCTAGTTTTAGAATA
TAAAATAAATCTTGCTAATAGTTTGAAAATTCTAAAATGGAGGGGTTTAC
T;
and

(SEQ ID NO: 26)

TAAAATTTATTTGTATACAGAGATTATCGCTTCTCGTCTATTGAGAAGTG
ATTTTTTTATCTTTCTGTATCCATAATGGGGAATTTTTTGTTTTTATATA
ATAGTAATACGGAAAAGATAGTTCTAATTCCCTAAAAAAATACACTTCAT
ATATTTTTAGTTTTGGTTATCGATTTGGCATGGAATTTGCATGTGTAAGA
AGGGTTTATCAGACATTTTAATTGTCAGGAAAAGAAAACTAGTTACATAC
TATAAAACTATTTTACATTATCTACTAGAATCAAAAAATTTTAATAATAT
AGTAAAATGTAGTTGACCTTCCTAGTTTTAGAATATAAAATAAATCTTGC
TAATAGTTTGAAAATTCTAAAATGGAGGGGTTTACT.

Transformants were grown overnight in semi-defined A5 media with 1% glucose. 250 μl of the overnight culture was used to inoculate 5 ml of fermentation media. The samples were fermented for 24 hrs in a u-24 device from Microreactor Technologies. CelA production was determined by pNPG activity assay as described above in Example 3. Table 4 illustrates the secretion of CelA protein under the control of the truncated inhA promoters as measured by pNPG hydrolysis.

TABLE 4
Secretion of CelA.

| | Fold Improvement over BmI positive |
|---|---|
| Truncated inhA promoter | control (±) |
| Control (BmI) | 1.00 (±.09) |
| BmI201 | 1.15 (±.14) |
| BmI351 | 0.90 (±.05) |
| BmI301 | 0.94 (±.04) |
| BmI386 | 0.96 (±.08) |

### Example 5

### Variant Promoters

Promoter libraries targeting (a) the region between −35 and −10 and the RBS and (b) the RBS and the start codon of SEQ ID NO: 1 were constructed using standard library construction techniques. (See Hemsley, A. et al., (1989) Nucleic Acids Res. 17(16):6545-51 and Matsumura, I. et al., (2001) Biotechniques 30(3):474-6).

The libraries were screened using either the celA or cbh construct described above which were transformed into the B. megaterium strain. Protein expression was measured by SDS-PAGE and/or enzymatic activity. Detailed descriptions of the assays are described above. Improved variants of the inhA promoter are listed in Table 5. SEQ ID NO: 1 further including the nucleotides GTACA at the 3′ end was used as the positive control (SEQ ID NO: 2). The variants listed in Table 5 are depicted in the 5′ to 3′ direction starting from the TATAAA box.

TABLE 5
InhA variant fold improvement over the positive
control (FI). The RBS for each sequence is in
bold and underlined. The start codon (ATG) is
in bold and the TATAAA box is in italics.

| SEQ ID | | |
|---|---|---|
| NO: | | FI |
| 27 | TATAAAATAAGGCATGCTAATAGTTTGAAAATTTAAAA | 1.5 |
| | TGGAGGGGCGAAGTGTACAATG | |
| 28 | TATAAAATAAGGCATGCTAATAGTTTG | 1.5 |
| | AAAATTCTAAAATGGAGGGGCTCGATGTACAATG | |
| 29 | TATAAAATAAGGCATGCTAATAGTTTGAAAATTCTAAA | 1.4 |
| | ATGGAGGGGCTCGATGTACAATG | |
| 30 | TATAAAATAAGGCATGCTAATAGTTTGAAAATTCTAAA | 1.4 |
| | ATGGAGGGGTTTATGGACCAATG | |
| 31 | TATAAAATAAGGCATGCTAATAGTTTGAAAATTCTAAA | 1.3 |
| | ATGGAGGGGCATTATGTACAATG | |
| 32 | TATAAAATAAGGCATGCTAATAGTTTGAAAATTCTAAA | 1.3 |
| | ATGGAGGGGTCCTATGTACAATG | |
| 33 | TATAAAATAAGGCATGCTAATAGTTTGAAAATTTAAGA | 1.1 |
| | GATGGAGGGATTCGTATG | |
| 34 | TATAAAATAAGGCATGCTAATAGTTTGAAAATTTGATA | 1.2 |
| | TTTGGAGGGAATAAAATG | |
| 35 | TATAAAATAAGGCATGCTAATAGTTTGAAAATTATGTA | 1.2 |
| | GGAGGGCCATACTCTATG | |
| 36 | TATAAAATAAGGCATGCTAATAGTTTGAAAATTCTAAAA | 1.4 |
| | TGGAGGGGTTTATGGGACAATG | |
| 37 | TATAAAATAAGGCATGCTAATAGTTTGAAAATTCTAAAA | 1.3 |
| | TGGAGGGGCAACATGTACAATG | |
| 38 | TATAAAATAAGGCATGCTAATAGTTTGAAAATTCTAAAA | 1.2 |
| | TGGAGGGGTCTTATGTACAATG | |
| 39 | TATAAAATAAGGCATGCTAATAGTTTGAAAATTCTAAAA | 1.2 |
| | TGGAGGGGTTTACTCCATGATG | |
| 40 | TATAAAATAAGGCATGCTAATAGTTTGAAAATTCTAAAA | 1.3 |
| | TGGAGGGGTTTACTGTACAATG | |
| 41 | TATAAAATATAGGCTGCTAATAGTTTGAAAATTCTAAAA | 1.2 |
| | TGGAGGGGTTTACTGTACAATG | |
| 42 | TATAAAATAAATCTTGCTAATAGTTTGAAAATTCTAAAA | 1.3 |
| | TGGAGGGGTTTACTGTACAATG | |
| 43 | TATAAAATAGGGTCTGCTAATAGTTTGAAAATTCTAAAA | 1.4 |
| | TGGAGGGGTTTACTGTACAATG | |
| 44 | TATAAAATAAATC - - - AATAGTTTGAAAATTCTAAA | 1.2 |
| | ATGGAGGGGTTTACTGTACAATG | |
| 45 | TATAAAATAATGACTGCTAATAGTTTGAAAATTCTAAAA | 1.3 |
| | TGGAGGGGTTTACTGTACAATG | |
| 46 | TATAAAATAAAGGCGGCTAATAGTTTGAAAATTCTAAAA | 1.2 |
| | TGGAGGGGTTTACTGTACAATG | |
| 47 | TATAAAATAAATCTGCCTCATAGTTTGAAAATTCTAAAA | 1.5 |
| | TGGAGGGGTTTACTGTACAATG | |
| 48 | TATAAAATAAATCTATTTTATAGTTTGAAAATTCTAAAAT | 2.0 |
| | GGAGGGGTTTACTGTACAATG | |
| 49 | TATAAAATAAATCTCCCCTATAGTTTGAAAATTCTAAAA | 2.5 |
| | TGGAGGGGTTTACTGTACAATG | |
| 50 | TATAAAATACGGGGTGCTAATAGTTTGAAAATTCTAAAA | 1.9 |
| | TGGAGGGGTTTACTGTACAATG | |
| 51 | TATAAAATAATCGATGCTAATAGTTTGAAAATTCTAAAA | 1.8 |
| | TGGAGGGGTTTACTGTACAATG | |
| 52 | TATAAAATAAATCTCCTGTATAGTTTGAAAATTCTAAAA | 1.9 |
| | TGGAGGGGTTTACTGTACAATG | |
| 53 | TATAAAATAAATCTTCGCCATAGTTTGAAAATTCTAAAA | 1.8 |
| | TGGAGGGGTTTACTGTACAATG | |
| 54 | TATAAAATAAATCTCCCCAATAGTTTGAAAATTCTAAAT | 1.9 |
| | GGAGGGGTTTACTGTACAATG | |
| 55 | TATAAAATACTCAGTGCTAATAGTTTGAAAATTCTAAAA | 1.9 |
| | TGGAGGGGTTTACTGTACAATG | |
| 56 | TATAAAATACTCAGTGCTAATAGTTTGAAAATTCTAAAA | 1.9 |
| | TGGAGGGGTTTACTGTACAATG | |
| 57 | TATAAAATAAATCTTCCAATAGTTTGAAAATTCTAAAAT | 1.4 |
| | GGAGGGGTTTACTGTACAATG | |
| 58 | TATAAAATATTCGGTGCTAATAGTTTGAAAATTCTAAAA | 1.9 |
| | TGGAGGGGTTTACTGTACAATG | |
| 59 | TATAAAATACAACTGCTAATAGTTTGAAAATTCTAAAAT | 1.8 |
| | GGAGGGGTTTACTGTACAATG | |
| 60 | TATAAAATAATCCGTGCTAATAGTTTGAAAATTCTAAAA | 1.8 |
| | TGGAGGGGTTTACTGTACAATG | |
| 61 | TATAAAATAAATCTCCTTGATAGTTTGAAAATTCTAAAA | 2.0 |
| | TGGAGGGGTTTACTGTACAATG | |
| 62 | TATAAAATACACATTGCTAATAGTTTGAAAATTCTAAAA | 1.8 |
| | TGGAGGGGTTTACTGTACAATG | |
| 63 | TATAAAATACCAATTGCTAATAGTTTGAAAATTCTAAAA | 1.8 |
| | TGGAGGGGTTTACTGTACAATG | |

What is claimed is:

1. An isolated promoter comprising,
a) a nucleotide sequence of SEQ ID NO: 1 or SEQ ID NO: 3;
b) a truncated nucleotide sequence of SEQ ID NO: 1 or SEQ ID NO: 3;
c) a nucleotide sequence having at least 90% sequence identity to a) or b); or
d) a variant nucleotide sequence or a truncated variant nucleotide sequence of SEQ ID NO: 1, wherein said variant or truncated variant has between 1 and 10 substitutions when aligned with SEQ ID NO: 1 or the corresponding truncated sequence thereof.

2. The isolated promoter of claim 1, wherein the promoter comprises the nucleotide sequence of SEQ ID NO: 1 or a sequence having at least 90% sequence identity thereto.

3. The isolated promoter of claim 1, wherein the promoter comprises a truncated nucleotide sequence of SEQ ID NO: 1 or a truncated nucleotide sequence having at least 90% sequence identity to SEQ ID NO: 1.

4. The isolated promoter of claim 3, wherein the truncated nucleotide sequence comprises the nucleotide sequences of SEQ ID NO: 4, SEQ ID NO: 5, SEQ ID NO: 6, SEQ ID NO: 7, SEQ ID NO: 23, SEQ ID NO: 24, SEQ ID NO: 25 or SEQ ID NO: 26.

5. The isolated promoter of claim 3, wherein the truncated nucleotide sequence includes truncation from the 5′ end of SEQ ID NO: 1 and includes about 200 nucleotides.

6. The isolated promoter of claim 1, wherein the promoter comprises a variant nucleotide sequence or a truncated variant nucleotide sequence of SEQ ID NO: 1 and wherein the variant or truncated variant has between 1 and 10 substitutions when aligned with SEQ ID NO: 1 or the truncated sequence thereof.

7. The isolated promoter of claim 1, wherein the nucleotide sequence has at least 95% sequence identity to the sequence of a) or b).

8. The isolated promoter of claim 1, wherein the nucleotide sequence has at least 97% sequence identity to the sequence of a) or b).

9. The isolated promoter of claim 1, wherein the variant nucleotide sequence comprises any one of the sequences selected from SEQ ID NOs: 27-63 when said sequence is aligned with the ribosome binding site of SEQ ID NO: 1 or SEQ ID NO: 23.

10. A vector comprising the isolated promoter of claim 1 operably linked to a heterologous coding sequence.

11. The vector of claim 10 further comprising a signal sequence.

12. The vector of claim 10, wherein the coding sequence codes for a protein.

13. The vector of claim 12, wherein the protein is an enzyme.

14. The vector of claim 13, wherein the enzyme is a glucoamylase, a protease, an alpha amylase, a cellulase, a hemicellulase, a xylanase, an esterase, a cutinase, a phytase, a lipase, an oxidoreductase, a laccase, an isomerase, a pullulanase, a phenol oxidizing enzyme, a mannase, a catalase, a glucose oxidase, a transferase or a lyase.

15. The vector of claim 14, wherein the enzyme is a cellulase.

16. The vector of claim 15, wherein the cellulase is an endoglucanase, a cellobiohydrolase or a β-glucosidase.

17. A transformed microbial host cell comprising the vector of claim 10.

18. The transformed microbial host cell of claim 17, wherein the transformed cell is a bacterial cell.

19. The transformed microbial host cell of claim 18, wherein the bacterial cell is a Bacillus cell or an E. coli cell.

20. A process for producing a protein in a host cell, comprising transforming a microbial host cell with the vector of claim 10 and culturing the transformed host cell under conditions permissible for the expression and production of the protein.

21. The process according to claim 20 further comprising recovering the protein.

22. The process of claim 21, wherein the protein is an enzyme.
